(19) United States
(12) Patent Application Publication
(10) Pub. No.: US 2023/0270342 A1
(43) Pub. Date: Aug. 31, 2023

(54) BIOLOGICAL INFORMATION MANAGEMENT SYSTEM

(71) Applicant: TERUMO KABUSHIKI KAISHA, Tokyo (JP)

(72) Inventors: Yusuke SEKINE, Chigasaki-shi (JP); Naoya SHIMADA, Hadano-shi (JP)

(73) Assignee: TERUMO KABUSHIKI KAISHA, Tokyo (JP)

(21) Appl. No.: 18/311,936

(22) Filed: May 4, 2023

Related U.S. Application Data
(63) Continuation of application No. PCT/JP2021/048540, filed on Dec. 27, 2021.

(30) Foreign Application Priority Data
Nov. 5, 2020 (JP) 2020-185153

(51) Int. Cl. A61B 5/01; A61B 5/00; A61B 5/0205; G16H 40/67

(52) U.S. Cl. CPC A61B 5/01; A61B 5/4875; A61B 5/6833; A61B 5/02055; G16H 40/67; A61B 5/4011; A61B 5/4017; A61B 2560/0252; A61B 2562/029; A61B 5/02438

(57) ABSTRACT

An attachment unit of the biological information management system includes a body temperature measurement unit that measures a body temperature of a subject on an underarm portion and a body water measurement unit that measures body water of the subject on the underarm portion, and the body temperature measurement unit and the body water measurement unit of the attachment unit are attached to the underarm portion. The biological information transmission unit sends the body temperature of the subject measured by the body temperature measurement unit and the body water of the subject measured by the body water measurement unit to a determination unit, and the determination unit outputs a determination result of whether or not there is a possibility that the subject develops infection or heatstroke based on information of the body temperature and body water of the subject sent from the biological information transmission unit.

### CROSS-REFERENCES TO RELATED APPLICATIONS

This application is a continuation of International Application No. PCT/JP2021/048540 filed on Dec. 27, 2021, which claims priority to Japanese Application No. 2020-185153 filed on Nov. 5, 2020, the entire content of both of which is incorporated herein by reference.

### TECHNOLOGICAL FIELD

The present disclosure generally relates to a biological information management system that monitors vital information of a body temperature and body water of a subject and determines infection, dehydration, and heatstroke of the subject by attaching an attachment unit including a body temperature measurement unit and a body water measurement unit to an underarm portion (axilla) of the subject.

### BACKGROUND DISCUSSION

For each individual such as a child or an aged person, a daily body temperature control is very difficult. Furthermore, in a case where infection caused by new coronavirus has occurred, each individual wears a mask or the like, and thus a child and an aged person are particularly likely to develop heatstroke or the like. Therefore, it is still more difficult to manage daily physical condition of each individual.

Furthermore, in a case where people live or act as a group together in a school, an elderly care facility, or the like, it is difficult to manage the daily physical condition of each individual, and daily health management of each individual is currently entrusted to each individual, a teacher, a care worker, or the like.

Moreover, in a case where people suffer when disaster frequently occurs as in recent years, people are forced to live together, and there is a possibility of mass infection in a situation where the physical condition of each individual deteriorates due to the disaster. Therefore, each individual has to pay more attention to management of the daily physical condition.

For example, even when each individual measures the body temperature and reports to the administrator by themselves, each individual cannot constantly measure the body temperature, and thus the administrator can only confirm the body temperature of each individual recorded in an analog manner at a certain time point. Therefore, it is insufficient for the administrator to manage the daily physical condition of each individual.

It is required to determine whether or not each individual develops infection or heatstroke on the premise that body temperature measurement is performed before the time of going out, before the time of engaging in activities at work or school, or before the time of meeting a person, regardless of age and gender, including infants, children, and aged people. However, since the result of the body temperature measurement is actually self-reported, the body temperature may be roughly measured, or the body temperature may not be actually measured.

Japanese Patent Application Publication No. 2020-89592 A discloses a heatstroke monitoring device. The heatstroke monitoring device determines whether or not a person is suspected of heatstroke based on detection results of a body water detection unit and a body temperature measurement unit. In this heatstroke monitoring device, the body water is detected based on a wavelength component of near-infrared light on a body surface of a person, and a body temperature is measured from an infrared ray emitted by the person.

The heatstroke monitoring device of Japanese Patent Application Publication No. 2020-89592 A collects information regarding body water and a body temperature of a person's body surface in a non-contact manner, and monitors heat stroke of a person passing through a gate. In a case where the heatstroke is suspected, a gate bar prevents the person from passing through the gate. However, since this heatstroke monitoring device is a fixed and large-scale device, the heatstroke cannot be constantly monitored for a plurality of individual persons before the subject goes out, before the subject engages in activities at work, kindergarten, nursery school, school, or the like, or before the subject meets a person, regardless of age and gender, including infants, children, and aged people, and further, monitoring of the infection is not described or suggested at all. Therefore, there is a problem that in a case where there is a change in physical condition, it is not possible to determine whether the cause is due to infection, heatstroke, or dehydration, and thus appropriate treatment cannot be performed, or even in a case where the physical condition deteriorates due to the heatstroke, treatment is hesitated due to fear of the infection.

### SUMMARY

The present disclosure relates to a biological information management system capable of determining whether or not each subject develops infection or heatstroke while continuously performing monitoring before the subject goes out, before the subject engages in activities at work or school, or before the subject meets a person, regardless of age and gender, including children and aged people.

According to an aspect of the present disclosure, there is provided a biological information management system including: an attachment unit that includes a body temperature measurement unit that measures a body temperature of a subject on an underarm portion of the subject and a body water measurement unit that measures body water of the subject on the underarm portion of the subject, in which the body temperature measurement unit and the body water measurement unit are attached to the underarm portion; a biological information transmission unit that transmits the body temperature of the subject measured by the body temperature measurement unit and the body water of the subject measured by the body water measurement unit; and a determination unit that outputs a determination result of whether or not there is a possibility that the subject develops infection or heatstroke, based on the body temperature of the subject and the body water of the subject, which are transmitted from the biological information transmission unit.

In the biological information management system according to the aspect of the present disclosure, since the attachment unit is attached to the underarm portion of the subject, the body temperature measurement unit and the body water measurement unit can be brought into contact with the skin on the underarm portion of the subject without causing discomfort, and the body temperature and body water of the subject can be continuously or sequentially measured on the underarm portion with accuracy. When the biological information transmission unit sends the measured body temperature and body water of the subject to the determination unit, the determination unit can output a determination result of whether or not there is a possibility that the subject develops infection or heatstroke based on the body temperature and body water of the subject. Therefore, the biological information management system can determine whether or not each subject develops infection or heatstroke while continuously and sequentially monitoring the body temperature and body water before the time of going out, before the time of engaging in activities at work or school, or before the time of meeting a person, regardless of age and gender, including children and aged people.

Since the symptoms of heatstroke (dehydration) and symptoms of infection change from moment to moment, the body temperature measurement unit and the body water measurement unit are disposed in the attachment unit, and the attachment unit is constantly attached to the underarm portion (armpit or axilla). Therefore, the body temperature and body water of the subject can be sequentially or constantly measured. As a result, the biological information management system can determine the possibility of heatstroke (dehydration) or infection at an earlier timing and with relatively higher accuracy.

In the biological information management system according to the aspect of the present disclosure, the determination unit preferably determines whether or not the subject develops dehydration in addition to the infection or the heatstroke.

In the biological information management system according to the aspect of the present disclosure, it can be determined whether or not the subject develops dehydration in addition to the infection or the heatstroke, while being continuously monitored, and thus the usefulness is further improved.

The biological information management system according to the aspect of the present disclosure preferably further includes: a recording unit that records the body temperature of the subject and the body water of the subject; and a notification unit that notifies the determination result in a case where the determination unit outputs the determination result indicating that there is a possibility that the subject develops the infection or the heatstroke.

In the biological information management system according to the aspect of the present disclosure, the recording unit records the body temperature of the subject and the body water of the subject. In a case where the determination unit outputs a determination result indicating that there is a possibility that the subject develops infection or heatstroke, the notification unit can notify the determination result, and thus the administrator can reliably obtain the determination result.

The biological information management system according to the aspect of the present disclosure preferably further includes a display unit that displays the determination result in a case where the determination unit outputs the determination result indicating that there is a possibility that the subject develops the infection or the heatstroke.

In the biological information management system according to the aspect of the present disclosure, in a case where the determination unit outputs a determination result indicating that there is a possibility that the subject develops infection or heatstroke, the display unit can display the determination result, and thus the administrator can reliably and visually obtain the determination result.

The biological information management system according to the aspect of the present disclosure preferably further includes a main body apparatus including the determination unit, in which the biological information transmission unit is disposed in the attachment unit, the attachment unit has identification information for identifying the subject, and the identification information is sent to the determination unit of the main body apparatus by the biological information transmission unit.

In the biological information management system according to the aspect of the present disclosure, since identification information of the subject is sent to the determination unit of the main body apparatus, the determination unit can reliably identify and specify who the subject is.

In the biological information management system according to the aspect of the present disclosure, the recording unit preferably includes: a primary information recording unit that records, as primary information obtained from the subject, the body temperature of the subject and the body water of the subject; a secondary information recording unit that records, as secondary information obtained from the subject, abnormality in taste and smell of the subject, and presence or absence of cough and dyspnea; and a tertiary information recording unit that records, as tertiary information, an external environment of a local place where the subject is present.

In the biological information management system according to the aspect of the present disclosure, the recording unit can record different types of information regarding the subject separately into the primary information, the secondary information, and the tertiary information. The biological information management system determines the possibility of heatstroke (dehydration) or infection in consideration of not only numerical values (primary information) such as the body temperature and body water of the subject but also actual symptoms (secondary information) of the subject and an external environment (tertiary information). Therefore, it is possible to increase the accuracy of determination for the heatstroke (dehydration) and the infection.

In the biological information management system according to the aspect of the present disclosure, the external environment of the local place where the subject is present preferably indicates whether or not the local place where the subject is present is a cluster region of the infection or a region around the cluster region, whether or not there is a possibility that the subject is a close contact of the infection, or a climate of the place where the subject is present.

In the biological information management system according to the aspect of the present disclosure, the determination unit can more accurately output the determination result of whether or not the local place where the subject is present is a cluster region of the infection or a region around the cluster region, the determination result of whether or not there is a possibility that the subject is a close contact of the infection, or the determination result of whether or not there is a possibility that the subject develops the infection and the heatstroke in a state in which a climate of the place where the subject is present is recognized.

In the biological information management system according to the aspect of the present disclosure, it is preferable that the determination unit determines whether or not the subject has a fever and whether or not the body water of the subject decreases, determines whether or not there is an abnormality in taste/smell, whether or not there is cough/dyspnea, and whether or not there is numbness in arms or legs or muscle spasms, and determines the external environment of the local place where the subject is present.

In the biological information management system according to the aspect of the present disclosure, the determination unit determines whether or not the subject has a fever and whether or not the body water of the subject decreases, and determines whether or not there is an abnormality in taste/smell, whether or not there is cough/dyspnea, and whether or not there is numbness in arms or legs or muscle spasms. Then, in a state in which the external environment of the local place where the subject is present is recognized, the determination unit can accurately output the determination result of whether or not there is a possibility that the subject develops the infection or the heatstroke.

The biological information management system according to the aspect of the present disclosure preferably further includes: an environmental temperature measurement unit that measures an environmental temperature and sends environmental temperature information to the determination unit; and an environmental humidity measurement unit that measures environmental humidity and sends environmental humidity information to the determination unit.

In the biological information management system according to the aspect of the present disclosure, the determination unit can recognize an environmental state based on the environmental temperature information measured by the environmental temperature measurement unit and the environmental humidity information measured by the environmental humidity measurement unit.

The biological information management system according to the aspect of the present disclosure preferably further includes a detection unit that detects a pulse rate and a heart sound of the subject.

In the biological information management system according to the aspect of the present disclosure, the determination unit can obtain the state of the subject by detecting the pulse rate and the heart sound of the subject.

In the biological information management system according to the aspect of the present disclosure, in order to determine an abnormality in smell of the subject, the main body apparatus preferably includes a smell generation unit that generates a smell for the subject.

In the biological information management system according to the aspect of the present disclosure, for example, in order to examine the possibility that the subject is infected with the new coronavirus, in a case where whether or not the subject has dysosmia is confirmed, the subject sniffs the smell from the smell generation unit, and thus the dysosmia of the subject can be confirmed.

In the biological information management system according to the aspect of the present disclosure, the determination unit preferably is capable of acquiring external information from an information terminal via an information communication network.

In the biological information management system according to the aspect of the present disclosure, since the determination unit can obtain necessary information from the external information terminal, the determination can be made more accurately.

In the biological information management system according to the aspect of the present disclosure, the attachment unit preferably includes an attachment sheet, the body temperature measurement unit and the body water measurement unit preferably are disposed on the attachment sheet, and the attachment unit preferably is attached to an underarm portion of the subject.

In the biological information management system according to the aspect of the present disclosure, the body temperature measurement unit and the body water measurement unit can be brought into contact with the skin on the armpit of the subject by using the attachment sheet and constantly disposed without causing discomfort, and the biological information of the body temperature and body water of the subject can be accurately sent to the determination unit continuously or sequentially.

In the biological information management system according to the aspect of the present disclosure, the determination unit preferably outputs a determination result of whether or not there is a possibility that the subject develops the infection based on a comparison result obtained by comparing the body temperature of the subject with a determination value of the body temperature and a comparison result obtained by comparing the body water of the subject with a determination value of the body water.

In the biological information management system according to the aspect of the present disclosure, the possibility of development of the infection can be accurately determined by whether or not there is a possibility that the subject develops the infection based on a comparison result obtained by comparing the body temperature of the subject with a determination value of the body temperature and a comparison result obtained by comparing the body water of the subject with a determination value of the body water.

According to the aspect of the present disclosure, there is provided a biological information management system capable of determining whether or not each subject develops infection or heatstroke while continuously or sequentially performing monitoring before the subject goes out, before the subject engages in activities at work or school, or before the subject meets a person, regardless of age and gender, including children and aged people.

According to an aspect of the present disclosure, a method for managing biological information is disclosed, which comprises: attaching a body temperature measurement unit and a body water measurement unit to an underarm portion of a subject; measuring a body temperature of the subject on the underarm portion of the subject with a body temperature measurement unit; measuring body water of the subject on the underarm portion of the subject with a body water measurement unit; transmitting the body temperature measured by the body temperature measurement unit and the body water measured by the body water measurement unit to a determination unit from a biological information transmission unit; and outputting a determination result of whether or not there is a possibility that the subject develops an infection or heatstroke, based on the body temperature of the subject and the body water of the subject transmitted from the biological information transmission unit.

### BRIEF DESCRIPTION OF THE DRAWINGS

FIG. 1 is a block diagram illustrating a biological information management system according to a preferred embodiment of the present disclosure.

FIGS. 2A and 2B are diagrams illustrating a preferred example of an attachment unit included in the biological information management system illustrated in FIG. 1.

FIGS. 3A and 3B are views illustrating an underarm portion of a subject.

FIG. 4 is a diagram illustrating symptoms of infection (new coronavirus: COVID-19) and heatstroke (including dehydration).

FIG. 5 is a flowchart illustrating a flow in which a determination unit determines a possibility of infection, heatstroke, or dehydration based on information regarding a fever and information regarding body water, which are primary information.

FIG. 6 is a flowchart illustrating a flow in which a determination unit determines a possibility of infection or heatstroke/dehydration based on information regarding symptoms of a subject, which is secondary information.

FIG. 7 is a flowchart illustrating a flow in which a determination unit determines a possibility of infection or heatstroke/dehydration based on external environmental information which is tertiary information.

### DETAILED DESCRIPTION

Set forth below with reference to the accompanying drawings is a detailed description of embodiments of a biological information management system that monitors vital information of a body temperature and body water of a subject and determines infection, dehydration, and heatstroke of the subject by attaching an attachment unit including a body temperature measurement unit and a body water measurement unit to an underarm portion (axilla) of the subject. In the drawings, similar components are denoted by the same reference signs, and the detailed description of the similar components will be appropriately omitted.

Outline of Biological Information Management System

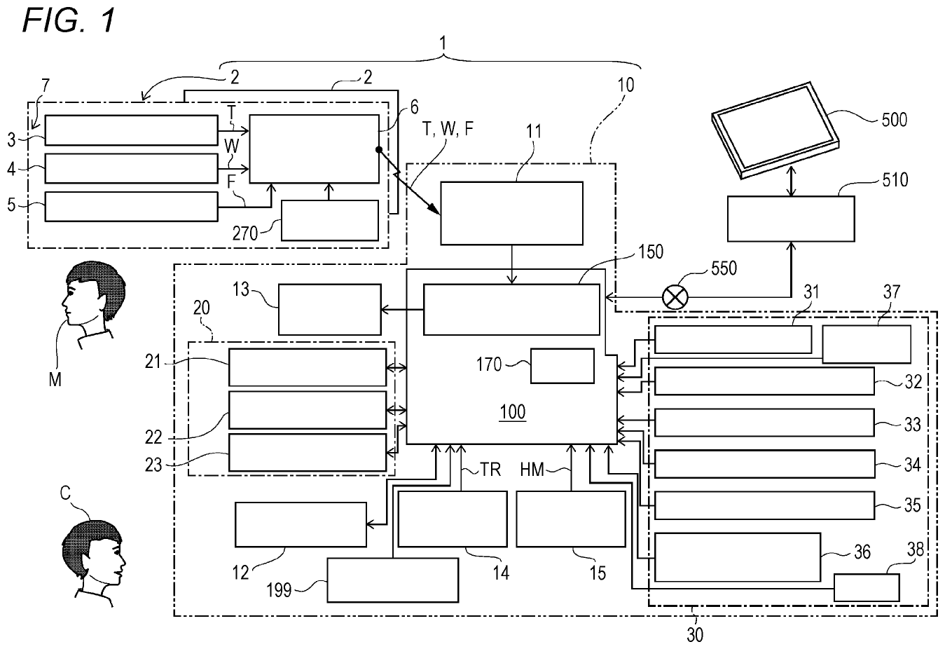

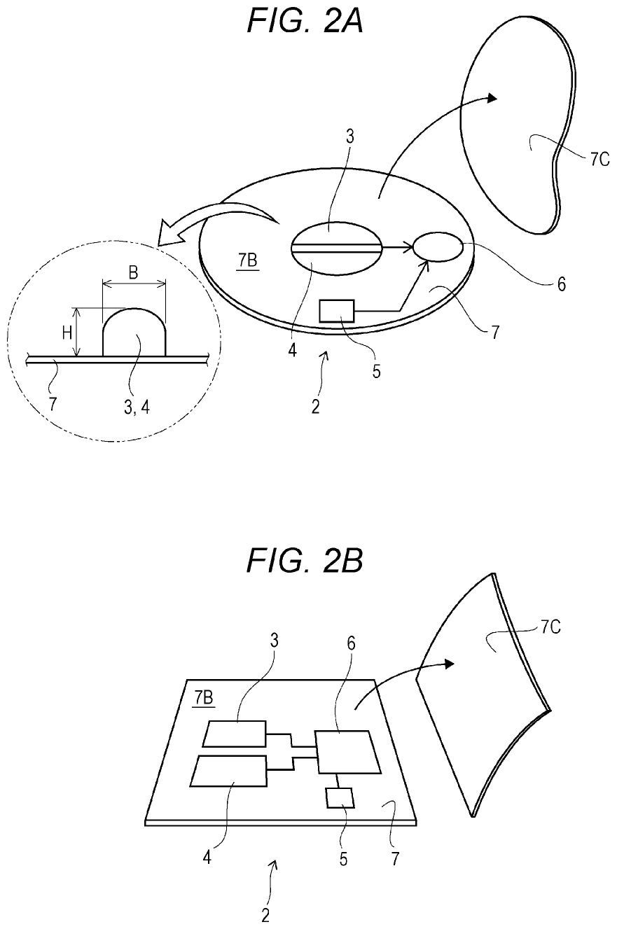

FIG. 1 is a block diagram illustrating a biological information management system 1 according to a preferred embodiment of the present disclosure. FIGS. 2A and 2B are diagrams illustrating a preferred example of an attachment unit 2 included in the biological information management system 1 illustrated in FIG. 1.

The biological information management system 1 illustrated in FIG. 1 includes the attachment unit 2 and a main body apparatus 10. The biological information management system 1 continuously and sequentially monitors the body temperature and body water of a subject M as vital values, and constantly determines the infection, dehydration, or heatstroke of the subject M by attaching a body temperature measurement unit 3 and body water measurement unit 4 of the attachment unit 2 to the underarm portion (axilla) of the subject M. The biological information management system 1 can determine whether or not each individual develops infection or heatstroke at any time while continuously or sequentially monitoring the body temperature and body water before the time of going out, before the time of engaging in activities at work or school, or before the time of meeting a person, regardless of age and gender, including children and aged people. Here, the continuous monitoring means that the subject M is constantly monitored for a predetermined period (for example, about one day to one month) and at predetermined time intervals (for example, at intervals of one to ten minutes).

Attachment Unit

The attachment unit 2 illustrated in FIG. 1 is attached to the underarm portion (axilla) of the subject M to measure the body temperature and body water of the subject M. The attachment unit 2 can be a disposable type vital measurement device that continuously or sequentially, and automatically measures the body temperature and the body water with relative accuracy. The attachment unit 2 includes an attachment sheet 7, and the attachment sheet 7 holds a body temperature measurement unit 3, a body water measurement unit 4, a subject identification information unit 5, and a biological information transmission unit 6. The biological information transmission unit 6 may be disposed not on the attachment sheet 7 but at a position different from the attachment sheet 7, for example, at another position of the body of the subject M.

The body temperature measurement unit 3 measures the body temperature of the subject M and sends body temperature information T to the biological information transmission unit 6. The body water measurement unit 4 measures the body water of the subject M and sends body water information W to the biological information transmission unit 6. The subject identification information unit 5 sends personal identification information F (for example, identification information for identifying an individual, such as a name and an age) of the subject M, which is recorded in advance, to the biological information transmission unit 6. The biological information transmission unit 6 transmits the body temperature information T and body water information W of the subject M and the personal identification information F of the subject M to a reception unit 11 of the main body apparatus 10 wirelessly or by using an infrared ray.

When the body temperature of the subject M is measured, a measurement site reflecting a deep temperature is considered to be a portion under the tongue, an armpit (axilla), or a rectum. In particular, even when the subject M is a child or an aged person, it is an “underarm portion (armpit or axilla)” for which continuous or sequential temperature measurement can be performed without causing discomfort.

Examples of a method for measuring the body water of the subject M include a blood test, a body composition meter, and palpation for examining the wet degree on the armpit. Among the methods described above, the blood test cannot be easily performed, and there is a time lag until a test result is obtained. In the case of using the body composition meter, the subject needs to stand on a scale or the like, and it is difficult to continuously monitor the body water. However, in order to examine the dehydration state of the aged person, palpation (i.e., pressing on the surface of the body) can be performed under the armpit, but this is to obtain only an analog numerical value, and when the result can be digitized, anyone can continuously or sequentially monitor the body water.

Therefore, from such a viewpoint, as the attachment unit 2, a relatively thin and light attachment unit 2 attached on the armpit of the subject M is employed, and the vital measurement can be performed constantly while simultaneously measuring the body temperature and the body water and continuously or sequentially monitoring the body temperature and the body water. An administrator C can quickly detect, for example, a fever, dehydration state, and heatstroke of the subject M with the main body apparatus 10 and relatively quickly take subsequent measures.

For example, even when the attachment unit 2 is attached to the armpit (axilla) of a child or an aged person, there is no sense of discomfort, and the body temperature and the body water can be automatically measured with relatively high accuracy. For example, in the case of a child who is the subject M, the attachment unit 2 can be used to continuously monitor the body temperature and the body water and rather quickly identify the subject M who may have a fever or get heatstroke. The attachment unit 2 can be attached to, for example, a child's armpit (axilla) and the child goes to school with the attachment unit 2 attached to the armpit, and there is no sense of discomfort in wearing the attachment unit 2 when the attachment unit 2 is attached. The attachment unit 2 can be easily made as a daily must-have, similarly to an action of wearing a hat or wearing a mask. Furthermore, since the attachment unit 2 does not interfere with the activity of the subject M and is not a portion that is always exposed like a portion around a hand or a face, the subject M does not need to be concerned with appearance.

The body temperature measurement unit 3 can relatively accurately measure the body temperature of the subject M by directly coming into contact with the skin on the armpit of the subject M, and for example, a sensor having a thermistor, a semiconductor temperature sensor, or the like can be adopted. The body water measurement unit 4 can accurately measure the body water of the subject M by directly coming into contact with the skin on the armpit of the subject M, and detects the body water of the subject M, for example, by detecting a physical quantity according to a supplied electric signal. For example, the body water measurement unit 4 detects the wet state of the skin on the armpit by measuring a change in capacitance as data related to water in a living body of the subject M, and detects the body water. The body water measurement unit 4 may measure not only capacitance but also impedance or perform optical measurement with an infrared ray sensor.

The body temperature measurement unit 3, the body water measurement unit 4, the subject identification information unit 5, and the biological information transmission unit 6 can be disposed on the attachment sheet 7 by, for example, a Micro-Electro-Mechanical systems (MEMS) technology. An integrated digital temperature and humidity sensor can be used as the body temperature measurement unit 3 and the body water measurement unit 4. With the MEMS technology, the digital temperature and humidity sensor fits into the recess portion of the armpit since the sensor unit is relatively thin even when the sensor unit is relatively rigid. Therefore, the subject M is less likely to feel the attachment sheet 7 when wearing, and has relatively little daily pain and discomfort from the attachment sheet 7 with MEMS technology. In a case where the attachment unit 2 does not include a power supply unit, for example, the biological information transmission unit 6 is configured to be capable of supplying power from the antenna unit by electromagnetic induction from the outside.

The size of the body temperature measurement unit 3 and the size of the body water measurement unit 4 are not particularly limited as long as the body temperature measurement unit 3 and the body water measurement unit 4 cause relatively little daily pain or discomfort and do not interfere with the activity. For example, in a case where the body temperature measurement unit 3 and the body water measurement unit 4 are configured as an integrated digital temperature and humidity sensor, for example, as illustrated in FIG. 2A, when a height direction from a measurement center point is set as H and a width is set as B, a height H can be preferably, for example, equal to or less than 3 cm. Moreover, when the height can be, for example, equal to or less than 1 cm, there is no sense of discomfort, and the subject does not feel that the attachment unit is attached. However, when the height H exceeds 3 cm, there is a possibility that the movable range of the arm is restricted, and discomfort or pain may be caused when the attachment unit is attached.

Furthermore, the width B can be, for example, preferably equal to or less than 3 cm. Moreover, when the height can be, for example, equal to or less than 2 cm, there is no sense of discomfort, and the subject does not feel that the attachment unit is attached. However, when the width B exceeds 3 cm, there is a possibility that the movable range of the arm can be restricted, and discomfort or pain may be caused when the attachment unit is attached.

FIGS. 2A and 2B illustrate shape examples of the attachment sheet 7 illustrated in FIG. 1. The attachment sheet 7 illustrated in FIG. 1 can have, for example, a shape as illustrated in FIGS. 2A and 2B, and for example, a medical/biological tape can be used. Since the attachment sheet 7 of the attachment unit 2 is attached to the body surface of the armpit, a surgical tape, a 3M tape (medical tape manufactured by 3M Japan Group), a wig tape, or the like can be used.

Since the armpit is a particularly delicate portion of the body, as the attachment sheet 7, it is necessary to employ a material that “is less likely to cause rash and not be noticeable” while maintaining the adhesiveness in order to correctly measure the body temperature and the body water. For example, as the attachment sheet 7, it is desirable to employ a material that is less likely to be stuffy (i.e., lacking ventilation), such as an attachment type material of an armpit sweat pad, a nonwoven Surgical Tape-21N (manufactured by NICHIBAN Co., Ltd.), and polyethylene having excellent moisture permeability. Furthermore, as attachment sheet 7, a material that can be repeatedly used without an adhesive remaining by being adhered to the skin with a gecko tape using van der Waals force, the gecko tape being manufactured, for example, by Nitto Denko Corporation. In a case where armpit hair is not grown or is relatively thin, a child and an aged person, and the like do not have to worry about the armpit hair, and even in a case where the armpit hair is grown, it is possible to reduce the influence of the armpit hair, for example, by shaving only the armpit hair of a portion to be subjected to attachment or selecting the above-described tape material.

The attachment sheet 7 illustrated in FIG. 2A is circular, and the attachment sheet 7 illustrated in FIG. 2B is rectangular. An adhesive layer 7B is provided on the surface of each attachment sheet 7, and a liner 7C is attached to the adhesive layer 7B.

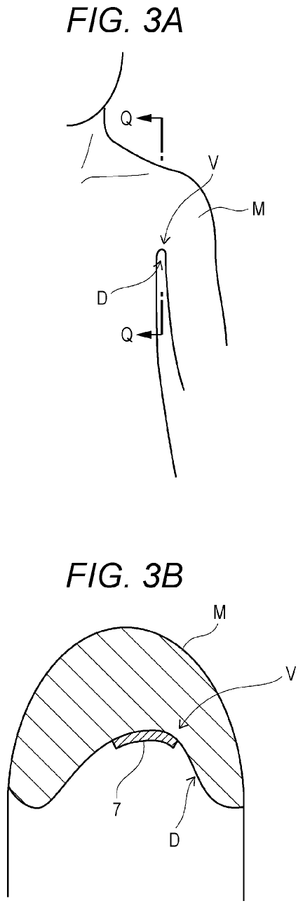

FIG. 3A illustrates an underarm portion V of the subject M, and FIG. 3B illustrates a cross section of the underarm portion V taken along line Q-Q in FIG. 3A. At the time of use, the liner 7C is removed from the adhesive layer 7B of the attachment sheet 7 illustrated in FIGS. 2A and 2B, and the adhesive layer 7B side illustrated in FIGS. 2A and 2B is detachably attached to the skin on an armpit D of the underarm portion V illustrated in FIG. 3B so as not to be peeled off for a relatively long time. Therefore, the body temperature measurement unit 3 and the body water measurement unit 4 are attached so as to be contact with the skin on the armpit D of the underarm portion V, and the body temperature and body water of the subject can be continuously monitored for a long time or sequentially monitored as necessary.

Main Body Apparatus

Next, a configuration example of the main body apparatus 10 will be described with reference to FIG. 1.

The reception unit 11 of the main body apparatus 10 illustrated in FIG. 1 receives the body temperature information T of the subject M, the body water information W, and the personal identification information F of the subject M from the biological information transmission unit 6 of the attachment unit 2 described above. The main body apparatus 10 can be used for management work such as notifying the administrator C, who has been given access right, of a health condition of the subject M, issuing a necessary warning, or acquiring necessary external information from the administrator C. The main body apparatus 10 can be placed, for example, in a management office or the like.

As illustrated in FIG. 1, the main body apparatus 10 can include a reception unit 11, a control unit 100, a display unit 12, a notification unit 13, an environmental temperature measurement unit 14, an environmental humidity measurement unit 15, a recording unit 20, and an input operation unit 30 for the administrator C to input various types of information. The control unit 100 includes a determination unit 150. Based on the biological information of the subject M such as the body temperature information T and body water information W of the subject M, the determination unit 150 can constantly determine whether or not the subject M develops infection, heatstroke, or dehydration while continuously performing monitoring before the subject M goes out, before the subject M engages in activities at work or school, or before the subject M meets a person. When a plurality of attachment units 2 are prepared, the number of the subjects M to be monitored is one or more, and the number is not particularly limited. In a case where there are a plurality of the subjects M, the biological information transmission unit 6 transmits the biological information to the reception unit 11 together with the personal identification information of the subject M.

The display unit 12 can display the biological information such as the body temperature information T and body water information W of the subject M, the personal identification information of the subject M, necessary notification, an alarm message, and the like according to a command of the control unit 100 as necessary. As the display unit 12, an organic electroluminescent (EL) display device, a liquid crystal display device, or the like can be adopted. The notification unit 13 performs notification or an alarm announcement with a buzzer sound, a voice announcement, or the like according to a command of the control unit 100 as necessary.

The environmental temperature measurement unit 14 measures the temperature of the environment in which the main body apparatus 10 is placed, and sends environmental temperature information TR to the control unit 100. The environmental humidity measurement unit 15 measures the humidity of the environment in which the main body apparatus 10 is placed, and sends environmental humidity information HM to the control unit 100. The temperature of the environmental and the humidity of the environmental in which the main body apparatus 10 is placed may be acquired from an external information terminal 500 via an information communication network 550 such as the Internet.

The recording unit 20 illustrated in FIG. 1 can include a primary information recording unit 21, a secondary information recording unit 22, and a tertiary (third) information recording unit 23. According to a command of the control unit 100, the primary information recording unit 21 records numerical information obtained from the subject M, for example, at least a body temperature (fever temperature) and body water, and also records presence or absence of a fever, presence or absence of a decrease in the body water, and the like as necessary. The body temperature of the subject M is obtained from the body temperature information T of the subject M, and the body water of the subject M is obtained from the body water information W of the subject M.

The secondary information recording unit 22 records the actual symptoms of the subject M. As the actual symptoms, information and events regarding the subject M, which are obtained by the administrator C performing hearings for the subject M, for example, at least an abnormality in taste and smell, and the presence or absence of cough and dyspnea are recorded, and as necessary, the presence or absence of numbness in arms or legs, and presence or absence of muscle spasms are recorded.

The tertiary information recording unit 23 records external environmental information of the subject, for example, indexes of whether or not a local environment in which the subject M is present is around a cluster region or whether or not there is a possibility that the subject M is a close contact, a local climate where the subject M is present, and whether or not the subject is likely to develop heatstroke/dehydration.

The input operation unit 30 illustrated in FIG. 1 can employ, for example, a graphical user interface (GUI), and the administrator C can arbitrarily input necessary information to the determination unit 150 of the control unit 100 by using the input operation unit 30.

The input operation unit 30 can include, for example, a dysgeusia confirmation input unit 31, a dysosmia determination smell generation unit 32, a dysosmia confirmation input unit 33, a confirmation input unit 34 for numbness in arms or legs and muscle spasms, a cluster region confirmation input unit 35, a confirmation input unit 36 for a possibility that a subject is a close contact, a cough/dyspnea confirmation input unit 37, and an input unit 38. The cough/dyspnea confirmation input unit 37 is a sound collection unit like a stethoscope for collecting sounds of cough and dyspnea in order to determine the cough and dyspnea by using a respiratory sound. The input unit and the dysosmia determination smell generation unit 32 can be optionally set in the main body apparatus 10, and may not be mounted in a case where they are unnecessary.

In the case of infection caused by the new coronavirus, the subject M may have a reduced taste sense. Therefore, in a case where the administrator C performs hearings for the subject M and confirms that the subject M has dysgeusia, the administrator C turns on the dysgeusia confirmation input unit 31. As a result, the determination unit 150 of the control unit 100 receives a signal from the dysgeusia confirmation input unit 31 and recognizes that the subject M is experiencing dysgeusia.

Furthermore, in the case of infection caused by the new coronavirus, the subject M may have a reduced olfactory sense. Therefore, in a case where the administrator C performs hearings for the subject M and confirms that the subject M has dysosmia (i.e., a change in sense of smell), the administrator C turns on the dysosmia confirmation input unit 33. As a result, the determination unit 150 of the control unit 100 receives a signal from the dysosmia confirmation input unit 33 and recognizes that the subject M is experiencing dysosmia.

In order for the administrator C to confirm this dysosmia, the dysosmia determination smell generation unit 32 is prepared. The dysosmia determination smell generation unit 32 is turned on by the administrator C in a case where it is desired to confirm whether the subject M has the dysosmia. As a result, the dysosmia determination smell generation unit 32 generates an arbitrary type of smell, and the subject M sniffs this smell. As a result, the administrator C performs hearings for the subject M and can confirm whether or not the subject M has the dysosmia. When the administrator C can confirm that the subject M has the dysosmia, the administrator C turns on the dysosmia confirmation input unit 33, and the determination unit 150 of the control unit 100 recognizes that the subject M is experiencing the dysosmia.

In a case where the administrator C performs hearings for the subject M and confirms that the subject M has numbness in arms or legs or muscle spasms, the administrator C turns on the confirmation input unit 34 for numbness in arms or legs and muscle spasms illustrated in FIG. 1. As a result, the determination unit 150 of the control unit 100 recognizes that the subject M is experiencing the numbness in arms or legs and the muscle spasms.

In a case where the place where the subject M is present is, for example, a cluster region of the infection caused by the new coronavirus or a region around the cluster region, the administrator C turns on the cluster region confirmation input unit 35. As a result, the determination unit 150 of the control unit 100 recognizes that the place where the subject M is present is the cluster region or a region around the cluster region. In a case where there is a possibility that the subject M is a close contact of infection caused by the new coronavirus, the administrator C turns on the confirmation input unit 36 for a possibility that a subject is a close contact. As a result, the determination unit 150 of the control unit 100 recognizes that there is a possibility that the subject M is a close contact.

As illustrated in FIG. 1, the determination unit 150 of the control unit 100 is preferably connected to the external information terminal 500 and a server 510 of the external information terminal 500 via the information communication network 550 such as the Internet. The information terminal 500 can be, for example, a tablet terminal, a portable computer, or the like, and the determination unit 150 of the control unit 100 can acquire various kinds of reference information from the information terminal 500. The various kinds of reference information are, for example, various kinds of real-time information such as information regarding infection caused by the new coronavirus, information regarding cluster occurrence, and weather information such as temperature and humidity in weather.

Meanwhile, as illustrated in FIG. 1, a detection unit 270 that detects a pulse rate and a heart sound may be disposed in the attachment unit 2. The detection unit 270 transmits information obtained by detecting the pulse rate and heart sound of the subject M from the biological information transmission unit 6 to the reception unit 11. Therefore, the determination unit 150 can capture dyspnea, palpitation, pneumonopathy, and the like of the subject M. The heart sound can be rather easily acquired by attaching the attachment unit 2 to the armpit although the attachment unit 2 is not attached to the chest.

In a case where sound detection is performed by the Korotkoff method (by detecting a blood pressure value and a pulse rate) in order to detect the pulse rate and the heart sound, either a microphone or a condenser microphone can be used as the detection unit. In this case, the detection unit 270 may be disposed in the attachment unit 2 or may be disposed at a position other than the attachment unit 2.

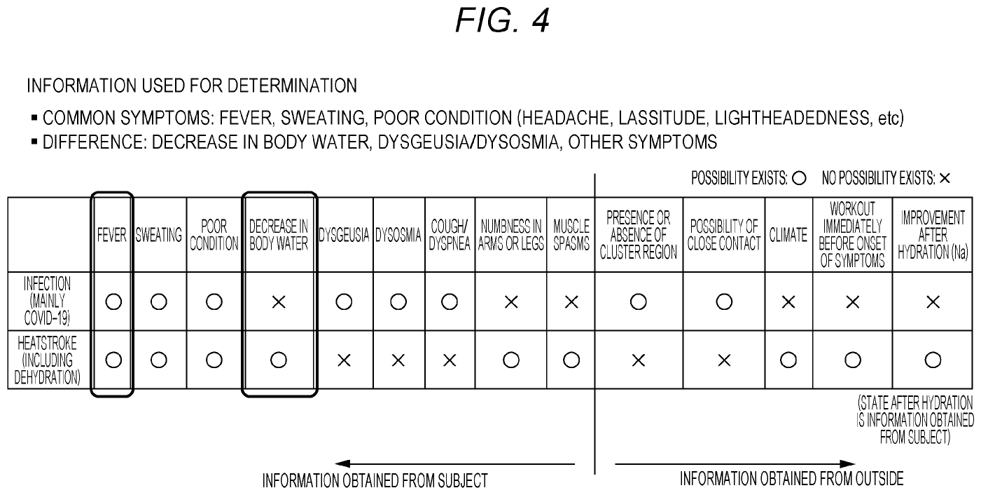

Next, information used for determination of the determination unit 150 illustrated in FIG. 1 will be described with reference to FIG. 4. FIG. 4 illustrates symptoms of infection (new coronavirus: COVID-19) and heatstroke (including dehydration). As illustrated in FIG. 4, common symptoms of the infection and the heatstroke are a “fever”, “sweating”, and a “poor condition (headache, lassitude, lightheadedness, and the like)”. Symptoms that are different between the infection and the heatstroke are a “decrease in body water”, “dysgeusia/dysosmia”, and “other symptoms”.

Each biological information item of a “fever”, “sweating”, a “bad condition”, “body water”, “dysgeusia”, “dysosmia”, “cough/dyspnea”, “numbness in arms or legs”, and “muscle spasms” is information obtained from the subject. Each information item of the “presence or absence of a cluster region”, a “possibility of close contact”, a “climate”, a “workout immediately before the onset of symptoms”, and “improvement after hydration (Na)” is information obtained from the outside. However, the information item “improvement after hydration (Na)” is information obtained from the subject M.

Description of Determination Operation Flow (1)

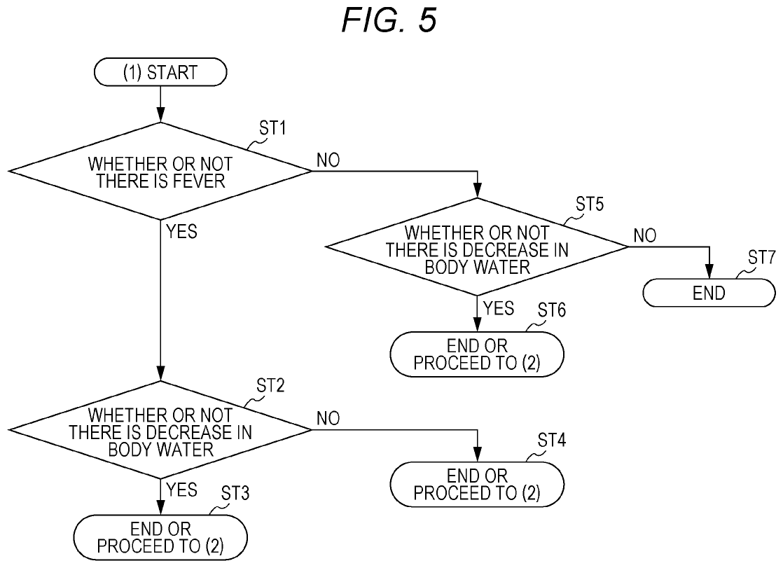

Next, a flow in which the determination unit 150 illustrated in FIG. 1 performs determination will be described with reference to FIG. 5.

Determination Flow (1)

In ST1 to ST7 of the determination flow (1) illustrated in FIG. 5, the determination unit 150 determines the possibility that the subject M develops infection or heatstroke/dehydration based on information regarding a fever and information regarding body water which are primary information.

In ST1 of the determination flow (1) illustrated in FIG. 5, the determination unit 150 illustrated in FIG. 1 determines whether or not the subject M has a “fever” based on the body temperature information T of the subject M obtained from the body temperature measurement unit 3. A reference value of the body temperature for which the determination unit 150 determines that the subject M has a “fever” can be, for example, equal to or greater than 37° C. The reference value is a normal temperature value (predetermined value) of the subject M, and is not limited to 37° C., and may be, for example, equal to or greater than 37.5° C. Furthermore, the reference value of the body temperature for determining that there is a “fever” may be set based on the normal temperature of the subject M, that is, an average value of the body temperature of the subject in a certain period of time in the past. With this setting, even a subject has a high normal temperature or even a subject has a low normal temperature, the presence or absence of a fever can be accurately determined. The administrator C confirms the body temperature information T of the subject M obtained from the body temperature measurement unit 3 by looking at the display unit 12 of the main body apparatus 10, and thus can check the “presence or absence of a fever” as to whether or not the subject M has a “fever”.

In ST1, when determining that the subject M has a “fever” (YES), the determination unit 150 proceeds to ST2. In ST2, the determination unit 150 checks “whether or not the body water of the subject M decreases”. The determination unit 150 determines whether or not the “body water of the subject M decreases” based on the body water information W of the subject M obtained from the body water measurement unit 4. A determination value at which the determination unit 150 determines that the “the body water of the subject M decreases” can be, for example, 98% of the reference value. The reference value is an average value of the body water of the subject M, and the determination value is not limited to 98% and may be, for example, 95% or 85%. Furthermore, a standard value of the body water may be set like 37° C. representing the presence of heat for the body temperature. In ST2, “whether or not the body water decreases” preferably can be determined more quickly by making time intervals shorter than the time intervals of monitoring in ST1.

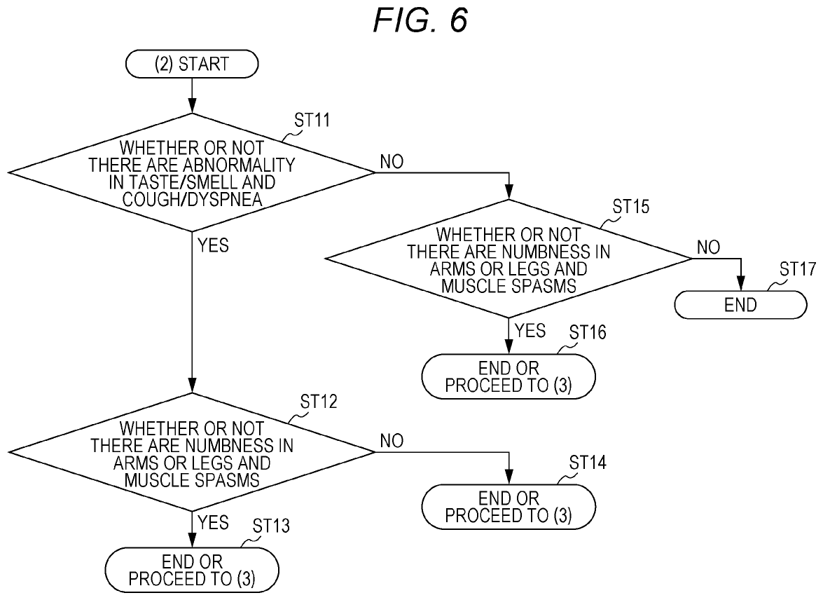

In ST2, when the determination unit 150 determines that the body water of the subject M is less than 98% of the reference value and the “body water of the subject M decreases” (YES), the determination unit 150 proceeds to ST3. In ST3, since the “body water of the subject M has already decreased”, the determination unit 150 causes the notification unit 13 to notify that “there is a possibility that the subject M develops the heatstroke” and causes the display unit 12 to display that. The determination processing may be ended in ST3, and moreover the determination processing may be continued by proceeding to ST11 of a determination flow (2) illustrated in FIG. 6.

On the other hand, in ST2, when the determination unit 150 determines that the body water of the subject M exceeds 98% of the reference value and the “body water of the subject M does not decrease” (NO), the determination unit 150 proceeds to ST4. In ST4, since it is already known that the subject M has a “fever” although the “body water of the subject M does not decrease”, the determination unit 150 also causes the notification unit 13 to notify that “there is a possibility that the subject M develops the infection” and causes the display unit 12 to display that. The determination processing may be ended in ST4, and moreover the determination processing may be continued by proceeding to ST11 of the determination flow (2) illustrated in FIG. 6.

On the other hand, returning to ST1, when determining that the subject M does not have a “fever” (NO), the determination unit 150 proceeds to ST5. In ST5, when the determination unit 150 determines that the body water of the subject M is less than 98% of the reference value and the “body water of the subject M decreases”, the determination unit 150 proceeds to ST6. In ST6, the determination unit 150 causes the notification unit 13 to notify that the “body water of the subject M decreases” and causes the display unit 12 to display that. The determination processing may be ended in ST6, and moreover the determination processing may be continued by proceeding to ST11 of the determination flow (2) illustrated in FIG. 6. In ST5, “whether or not the body water decreases” can be determined more quickly by making time intervals shorter than the time intervals of monitoring in ST1.

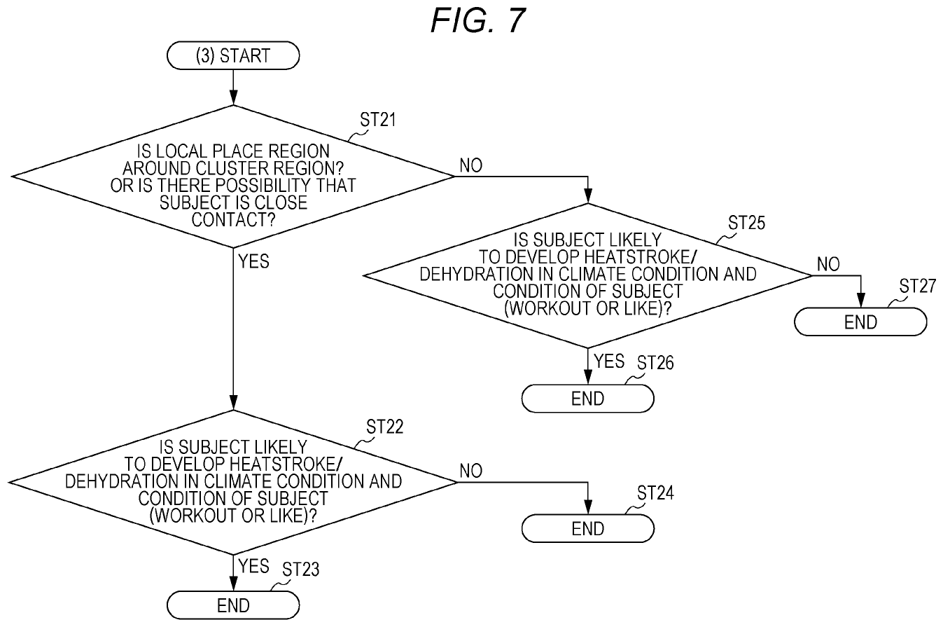

In ST5, when the determination unit 150 determines that the body water of the subject M exceeds 98% of the reference value and the “body water of the subject M does not decrease”, the determination unit 150 proceeds to ST7. In ST7, the determination processing is ended here. As a result, since the possibility that the subject M develops infection, as well as heatstroke and dehydration, is relatively low, the determination unit 150 does not cause the notification unit 13 to perform notification, but causes the display unit 12 to displays that the possibility of the infection, as well as the heatstroke and the dehydration, is relatively low. In ST7, since there is a possibility that the subject M develops infection, but has no symptoms, the processing may directly proceed to ST11 of the determination flow (2) illustrated in FIG. 6 or proceed to ST21 of a determination flow (3) illustrated in FIG. 7 to continuously perform the determination processing.

Determination Flow (2)

In ST11 to ST17 of the determination flow (2) illustrated in FIG. 6, the determination unit 150 determines the possibility that the subject M develops infection or heatstroke/dehydration based on information regarding symptoms of the subject M, which is secondary information.

In ST11 of the determination flow (2) illustrated in FIG. 6, the determination unit 150 illustrated in FIG. 1 determines an “abnormality in taste and smell” and the presence or absence of “cough/dyspnea” of the subject M. For example, when the administrator C asks the subject M about a current health condition and finds out that there is an abnormality in taste of the subject M, the administrator C turns on the dysgeusia confirmation input unit 31 illustrated in FIG. 1. As a result, the determination unit 150 recognizes that there is an “abnormality in taste” of the subject M, and the determination unit 150 causes the notification unit 13 to notify that there is an “abnormality in taste” of the subject M and causes the display unit 12 to display that.

Similarly, in ST11, the administrator C asks the subject M about the current health condition and finds out whether or not there is an abnormality in smell of the subject M. In this case, by generating a predetermined smell from the dysosmia determination smell generation unit 32 and causing the subject M to sniff the smell, the administrator C can reliably confirm whether or not there is an abnormality in smell of the subject M. When the administrator C finds out that the subject M has an abnormality in smell, the administrator C turns on the dysosmia confirmation input unit 33 illustrated in FIG. 1. As a result, the determination unit 150 recognizes that there is an “abnormality in smell” of the subject M, and the determination unit 150 causes the notification unit 13 to notify that there is an “abnormality in smell” of the subject M and causes the display unit 12 to display that.

Moreover, in ST11, when the administrator C asks the subject M about a current health condition and finds out that the subject M experiences the cough and dyspnea, the administrator C turns on the cough/dyspnea confirmation input unit 37 illustrated in FIG. 1. As a result, the determination unit 150 determines that the subject M experiences “cough and dyspnea”, and the determination unit 150 causes the notification unit 13 to notify that the subject M experiences “cough and dyspnea” and causes the display unit 12 to display that.

In this way, when the subject M experiences at least one of an “abnormality in taste and smell” or “cough and dyspnea” (YES), the processing proceeds to ST12. In ST12, the determination unit 150 illustrated in FIG. 1 determines whether or not the subject M experiences “numbness in arms or legs or muscle spasms”. When the administrator C asks the subject M about a current health condition and finds out that the subject M experiences numbness in arms or legs or muscle spasms, the administrator C turns on the confirmation input unit 34 for numbness in arms or legs and muscle spasms illustrated in FIG. 1. As a result, the determination unit 150 recognizes that the subject M is experiencing the numbness in arms or legs and the muscle spasms.

In this case, the processing proceeds from ST12 to ST13 (YES). The determination processing may be ended in ST13, and moreover the determination processing may be continued by proceeding to ST21 of the determination flow (3) illustrated in FIG. 7. In ST13, since the subject M experiences the “numbness in arms or legs and muscle spasms”, the determination unit 150 causes the notification unit 13 to notify that “there is a possibility that the subject M develops the heatstroke” and the subject M is “suspected of the infection” and causes the display unit 12 to display that.

In ST12, when the determination unit 150 of FIG. 1 determines that the subject M does not experience the “numbness in arms or legs and muscle spasms” (NO), the processing proceeds to ST14. In ST14, the subject M has no “possibility of heatstroke”, but is found to experience at least one of an “abnormality in taste and smell” or “cough and dyspnea”. Therefore, the determination unit 150 causes the notification unit 13 to notify that the subject M has a “possibility of the infection” and causes the display unit 12 to display that. The determination processing may be ended in ST14, and moreover the determination processing may be continued by proceeding to ST21 of the determination flow (3) illustrated in FIG. 7.

On the other hand, returning to ST11, in a case where the subject M does not experience any of the “abnormality in taste and smell” and the “cough and dyspnea” (NO), the processing proceeds to ST15. In ST15, as in ST12, the determination unit 150 illustrated in FIG. 1 determines whether or not the subject M experiences “numbness in arms or legs or muscle spasms”. As a result, when the subject M experiences the numbness in arms or legs or the muscle spasms (YES), the processing proceeds to ST16. The determination processing may be ended in ST16, and moreover the determination processing may be continued by proceeding to ST21 of the determination flow (3) illustrated in FIG. 7. In ST16, the determination unit 150 causes the notification unit 13 to notify that the subject M has a “possibility of dehydration/heatstroke” and causes the display unit 12 to display that the subject M has the “possibility of dehydration/heatstroke”.

In ST15, when the subject M does not experience the “numbness in arms or legs and muscle spasms” (NO), the processing proceeds to ST17, and the determination processing is ended. In ST17, since the possibility that the subject M develops infection, as well as heatstroke and dehydration, is relatively low, the notification unit 13 does not perform notification, but the display unit 12 displays that the possibility of the subject M developing infection, as well as heatstroke and dehydration is relatively low.

Determination Flow (3)

Next, in ST21 to ST27 of the determination flow (3) illustrated in FIG. 7, the determination unit 150 determines the possibility that the subject M develops infection or heatstroke/dehydration based on external environmental information which is tertiary information.

In ST21 illustrated in FIG. 7, the determination unit 150 illustrated in FIG. 1 determines whether a local place where the subject M is present is around the cluster region or whether there is a possibility that the subject M is a close contact. In this case, for example, the administrator C can confirm whether or not the local place where the subject M is present corresponds to a place where the cluster of the new coronavirus has occurred or the region around the cluster region by using the information terminal 500. In a case where the local place where the subject M is present corresponds to a place where the cluster of the new coronavirus has occurred or the region around the cluster region, the administrator C turns on the cluster region confirmation input unit 35 illustrated in FIG. 1.

As a result, the determination unit 150 recognizes that the local place where the subject M is present is the cluster region or a region around the cluster region. Alternatively, the administrator C operates the information terminal 500 to directly give, to the determination unit 150, information indicating that the local place where the subject M is present is the region where the cluster of the new coronavirus has occurred or the region around the region where the cluster of the coronavirus has occurred, and thus the determination unit 150 recognizes that the local place where the subject M is present is the cluster region or the region around the cluster region.

In ST21, for example, in a case where the administrator C finds out there is a possibility that the subject M is a close contact of the new coronavirus, the administrator C turns on the confirmation input unit 36 for a possibility that a subject is a close contact, which is illustrated in FIG. 1. As a result, the determination unit 150 recognizes that the subject M may be a close contact. In ST21, when it is determined that a local place where the subject M is present is a cluster region or the region around the cluster region, or there is a possibility that the subject M is a close contact (YES), the processing proceeds to ST22.

In ST22, it is determined whether or not the subject M is likely to develop heatstroke or dehydration in the climate condition and the condition (for example, during a workout or the like) of the subject M. For example, the environmental temperature measurement unit 14 illustrated in FIG. 1 sends the environmental temperature information TR to the determination unit 150 of the control unit 100, and the environmental humidity measurement unit 15 sends the environmental humidity information HM to the determination unit 150 of the control unit 100. As a result, the determination unit 150 recognizes a climate state for the temperature and humidity. When the administrator C asks the subject M about the current health condition to see whether the subject M is likely to develop heatstroke or dehydration and determines that the subject M is likely to develop the heatstroke or the dehydration, the administrator C turns on the input unit 38 illustrated in FIG. 1. As a result, the determination unit 150 recognizes that the subject M is likely to develop the heatstroke or the dehydration.

In ST22, when recognizing that the subject M is likely to develop the heatstroke or the dehydration (YES), the determination unit 150 proceeds to ST23. In ST23, there is a possibility that the subject M develops heatstroke/dehydration or infection, but the determination unit 150 causes the notification unit 13 to notify one of heatstroke/dehydration or infection which has a relatively higher possibility from the overall viewpoint, causes the display unit 12 to display that the subject M has a possibility of the one of heatstroke/dehydration or infection, and then ends the determination processing.

On the other hand, in ST22, when determining that the subject M is unlikely to develop the heatstroke or the dehydration (NO), the determination unit 150 proceeds to ST24 and ends the determination processing. In ST24, since there is a high possibility that the subject M develops the infection, the determination unit 150 causes the notification unit 13 to perform notification, causes the display unit 12 to display that there is a possibility that the subject M develops the infection, and ends the determination processing.

On the other hand, in ST21, when the determination unit 150 determines that the local place is not a cluster region and a region around the cluster region and there is a possibility that the subject M is not a close contact (NO), the processing proceeds to ST25. In ST25, as in ST22, the determination unit 150 illustrated in FIG. 1 determines whether or not the subject M is likely to develop the heatstroke or the dehydration in the climate condition and the condition (workout or the like) of the subject M. When the determination unit 150 recognizes that the subject M is likely to develop the heatstroke or the dehydration (YES), the processing proceeds to ST26.

In ST26, since there is a possibility that the subject M develops the heatstroke or the dehydration, the determination unit 150 causes the notification unit 13 to perform notification, causes the display unit 12 to display that there is a possibility that the subject M develops heatstroke or dehydration, and ends the determination processing. In ST25, when recognizing that the subject M is unlikely to develop the heatstroke or the dehydration (NO), the determination unit 150 proceeds to ST27 and ends the determination processing. In ST27, also taking into consideration results from the determination flows (1) and (2) illustrated in FIGS. 5 and 6 so far, the determination unit 150 causes the notification unit 13 to notify one having a relatively higher possibility of the infection or the heatstroke/dehydration from the overall viewpoint, cause the display unit 12 to display that one other than the subject M has a relatively higher possibility of infection or heatstroke/dehydration, and ends the determination processing. Alternatively, when comprehensively determining that there is a low possibility that the subject M develops infection, heatstroke, or dehydration, the determination unit 150 does not cause the notification unit 13 to perform notification, but causes the display unit 12 to display that there is a relatively low possibility that the subject M develops infection, heatstroke, or dehydration, and ends the determination processing.

The system of the portable or stationary main body apparatus 10 illustrated in FIG. 1 has an artificial intelligence (Al) function, and the main body apparatus 10 itself can acquire various types of information from the attachment unit 2, the recording unit 20, or the external information terminal 500 and performs determination. Furthermore, positional information may be acquired by a global positioning system (GPS) 170 built in the main body apparatus 10, and the acquired positional information may be used to specify and determine an occurrence position of a cluster of the infection. The GPS may be disposed in the attachment unit 2.

As items determined by the determination unit 150, the items can include a climate, a time (season), a temperature, humidity, wet bulb globe temperature (WBGT), and the like, and there are a situation in which the subject is working out, a situation in which the subject is not hydrated, and the like. Furthermore, the determination flow may include “hydrating the subject with +Na and watching the situation”. As a result, in a case where the body water of the subject M increases and symptoms of the heatstroke and symptoms of dehydration are also improved, it can be determined that the subject M is experiencing the heat stroke/dehydration. In a case where no improvement is seen, the subject M is suspected of the infection.

Note that communication between the reception unit 11 of the main body apparatus 10 and the biological information transmission unit 6 of the attachment unit 2 can be appropriately selectively performed according to a communication distance based on “IrDA standard” which is an international standard. For example, communication by the NFC method is performed in a case where the communication is performed at a distance of up to about 10 cm, and infrared ray communication and communication with Bluetooth® are performed. Note that, in the infrared communication or Bluetooth (Bluetooth), it is preferable that the attachment unit 2 is provided with a small button battery or the like used for the power supply unit.

In the determination flows (1) to (3) illustrated in FIGS. 5 to 7, when the notification unit 13 illustrated in FIG. 1 notifies the determination result, the main body apparatus 10 illustrated in FIG. 1 desirably induces (guides) a necessary treatment for the subject according to the determination result. For example, in the guidance example according to the determination result for the subject, for example, for the administrator C, a message may be displayed on the display unit 12 illustrated in FIG. 1 or a guidance message may be notified by the notification unit 13 with voice. Examples of the guidance message for the determination result for the subject M include “make the subject take a rest in the infirmary”, “call an ambulance (also report to the ambulance by using the telephone function 199 of the main body apparatus 10)”, “make the subject return home”, “call out to the subject since there is no subjective symptoms”, “encourage hydration”, “contact the health care center (also report to the health care center by using the telephone function 199 of the main body apparatus 10)”, and “follow the heatstroke prevention guidelines”.

Furthermore, the control unit 100 illustrated in FIG. 1 may transmit data of the body temperature and body water of the subject M obtained from the primary information recording unit 21 to the external information terminal 500 via the information communication network 550, and cause the information terminal 500 to perform the work of aggregating the data of the body temperature and body water of the subject M and the notification on an abnormal value.

In the biological information management system 1 according to the embodiment of the present disclosure described above, the body temperature and body water of the subject M can be continuously or sequentially monitored, and a heatstroke status and a dehydration status can be confirmed by measuring the body water. Therefore, the administrator C can rather quickly manage the condition of the subject M having dehydration symptoms. In addition, it may be difficult for a child or an aged person to manage the physical condition by himself or herself, but the physical condition of the subject M can be managed without troubling the administrator C. When disaster occurs, the body temperature and the dehydration status of the subject M can be managed even in a situation where the subject M is not be hydrated in order, for example, to avoid frequently going to the toilet. Regarding the daily body temperature management, the biological information management system 1 can continuously or sequentially monitor the troublesome daily body temperature measurement for the subject M automatically at all times without being conscious, and thus it is also a heatstroke countermeasure in summer.

Since the attachment unit 2 is attached to the underarm portion of the subject M, the body temperature measurement unit 3 and the body water measurement unit 4 can be brought into contact with the skin on the underarm portion of the subject M without causing discomfort, and the body temperature and body water of the subject M can be continuously or sequentially measured on the underarm portion with relative accuracy. When the biological information transmission unit 6 sends the measured body temperature and body water of the subject M to the determination unit 150, the determination unit 150 can output a determination result of whether or not there is a possibility that the subject M develops infection or heatstroke based on the body temperature and body water of the subject M.

It can be determined whether or not each subject M develops infection or heatstroke while being continuously or sequentially monitored before the subject M goes out, before the subject M engages in activities at work or school, or before the subject M meets a person, regardless of age and gender, including children and aged people. It can be determined whether or not the subject M develops dehydration in addition to the infection or the heatstroke, while being continuously or sequentially monitored, and thus the usefulness is further improved.

In a case where the determination unit 150 outputs a determination result indicating that there is a possibility that the subject M develops infection, heatstroke, or dehydration, the notification unit 13 can notify the determination result, and thus the administrator C can reliably obtain the determination result. In a case where the determination unit 150 outputs a determination result indicating that there is a possibility that the subject M develops infection, heatstroke, or dehydration, the display unit 12 can display the determination result, and thus the administrator C can reliably and visually obtain the determination result.

Since identification information of the subject M is sent to the determination unit 150 of the main body apparatus 10, the determination unit can reliably identify and specify who the subject is. The recording unit 20 can record different types of information regarding the subject separately into the primary information, the secondary information, and the tertiary information. The determination unit 150 can more accurately output the determination result of whether or not the local place where the subject M is present is a cluster region of the infection or a region around the cluster region, the determination result of whether or not there is a possibility that the subject M is a close contact of the infection, or the determination result of whether or not there is a possibility that the subject M develops the infection and the heatstroke in a state in which a climate of the place where the subject M is present is recognized.

The determination unit 150 determines whether or not the subject has a fever and whether or not the body water of the subject M decreases, and determines whether or not there is an abnormality in taste/smell, whether or not there is cough/dyspnea, and whether or not there is numbness in arms or legs or muscle spasms. Then, in a state in which the external environment of the local place where the subject M is present is recognized, the determination unit 150 can relatively accurately output the determination result of whether or not there is a possibility that the subject M develops the infection or the heatstroke. The determination unit 150 can recognize an environmental state based on the environmental temperature information TR measured by the environmental temperature measurement unit 14 and the environmental humidity information HM measured by the environmental humidity measurement unit 15.

For example, in order to examine the possibility that the subject M is infected with the new coronavirus, in a case where whether or not the subject M has dysosmia is confirmed, the subject M sniffs a smell from the dysosmia determination smell generation unit 32, and thus the dysosmia of the subject M can be confirmed. Since the determination unit 150 can obtain necessary information from the external information terminal 500, the determination can be made more accurately. The body temperature measurement unit 3 and the body water measurement unit 4 can be brought into contact with the skin on the armpit of the subject M by using the attachment sheet 7 and constantly disposed without causing discomfort, and the biological information of the body temperature and body water of the subject M can be accurately sent to the determination unit 150 continuously or sequentially.

Since the symptoms of heatstroke (dehydration) and infection change from moment to moment, at least the body temperature measurement unit 3 and the body water measurement unit 4 are disposed in the attachment unit 2, and the attachment unit 2 is constantly attached to the underarm portion (armpit or axilla). Therefore, the body temperature and body water of the subject M can be constantly measured sequentially or at predetermined time intervals. As a result, the biological information management system 1 can determine the possibility of heatstroke (dehydration) or infection at an earlier timing and with relatively higher accuracy. Therefore, a caregiver, a guardian, and the like, for example, can carefully care for and nurse the subject M, and it is possible to reduce or prevent secondary infections to the caregiver and the guardian due to viruses having strong infectivity and strong pathogenicity such as the new coronavirus and bacteria.

Furthermore, the biological information management system 1 determines the possibility of heatstroke (dehydration) or infection in consideration of not only numerical values (primary information) such as the body temperature and body water of the subject M but also actual symptoms (secondary information) of the subject M and an external environment (tertiary information). Therefore, it is possible to increase the accuracy of determination for the heatstroke (dehydration) and the infection. Therefore, a caregiver, a guardian, and the like, for example, can carefully care for and nurse the subject M, and it is possible to reduce or help prevent secondary infections to the caregiver and the guardian due to viruses having strong infectivity and strong pathogenicity such as the new coronavirus and bacteria.

The biological information management system 1 can cope with not only a case where the subject M is in the room but also a case where the subject M moves, for example, a case where the subject M goes out with an infant in a stroller or a case where the subject M goes out with an aged person on a wheelchair. Examples of the target facility to which the biological information management system 1 is applied can include a kindergarten, a nursery school, schools from an elementary school to a university, a protective institution, a nursing facility, a hospital, a company, a supermarket/retail store, a stadium, a live event venue, a movie theater, an ambulance, a ship, and an airplane, and these are not particularly limited. In particular, it is possible to bring about a great effect in an environment in which an unspecified large number of people gather, and an environment in which people are forced to gather such as a shelter in addition to a hard work/activity environment.

The detailed description above describes embodiments of a biological information management system that monitors vital information of a body temperature and body water of a subject and determines infection, dehydration, and heatstroke of the subject by attaching an attachment unit including a body temperature measurement unit and a body water measurement unit to an underarm portion (axilla) of the subject. The invention is not limited, however, to the precise embodiments and variations described. Various changes, modifications and equivalents may occur to one skilled in the art without departing from the spirit and scope of the invention as defined in the accompanying claims. It is expressly intended that all such changes, modifications and equivalents which fall within the scope of the claims are embraced by the claims.

What is claimed is:

1. A biological information management system comprising:
an attachment unit that includes a body temperature measurement unit configured to measure a body temperature of a subject on an underarm portion of the subject and a body water measurement unit configured to measure body water of the subject on the underarm portion of the subject, the body temperature measurement unit and the body water measurement unit being configured to be attached to the underarm portion of the subject;
a biological information transmission unit configured to transmit the body temperature of the subject measured by the body temperature measurement unit and the body water of the subject measured by the body water measurement unit to a determination unit; and
the determination unit being configured to output a determination result of whether or not there is a possibility that the subject develops an infection or heatstroke, based on the body temperature of the subject and the body water of the subject transmitted from the biological information transmission unit.

2. The biological information management system according to claim 1, wherein the determination unit is configured to determine whether or not the subject develops dehydration in addition to the infection or the heatstroke.

3. The biological information management system according to claim 1, further comprising:
a recording unit configured to record the body temperature of the subject and the body water of the subject; and
a notification unit configured to notify the determination result in a case where the determination unit outputs the determination result indicating that there is a possibility that the subject develops the infection or the heatstroke.

4. The biological information management system according to claim 1, further comprising:
a display unit configured to display the determination result in a case where the determination unit outputs the determination result indicating that there is a possibility that the subject develops the infection or the heatstroke.

5. The biological information management system according to claim 1, further comprising:
a main body apparatus including the determination unit, wherein the biological information transmission unit is disposed in the attachment unit, the attachment unit has identification information for identifying the subject, and the identification information is sent to the determination unit of the main body apparatus by the biological information transmission unit.

6. The biological information management system according to claim 3, wherein the recording unit includes:
a primary information recording unit configured to record, as primary information obtained from the subject, the body temperature of the subject and the body water of the subject;
a secondary information recording unit configured to record, as secondary information obtained from the subject, an abnormality in taste and smell of the subject, and presence or absence of cough and dyspnea; and
a tertiary information recording unit configured to record, as tertiary information, an external environment of a local place where the subject is present.

7. The biological information management system according to claim 6, wherein the external environment of the local place where the subject is present indicates whether or not the local place where the subject is present is a cluster region of the infection or a region around the cluster region, whether or not there is a possibility that the subject is a close contact of the infection, or a climate of the place where the subject is present.

8. The biological information management system according to claim 7, wherein the determination unit is configured to:
determine whether or not the subject has a fever and whether or not the body water of the subject decreases;
determine whether or not there is an abnormality in taste/smell, whether or not there is cough/dyspnea, and whether or not there is numbness in arms or legs or muscle spasms; and
determine the external environment of the local place where the subject is present.

9. The biological information management system according to claim 1, further comprising:
an environmental temperature measurement unit configured to measure an environmental temperature and send environmental temperature information to the determination unit; and
an environmental humidity measurement unit configured to measure environmental humidity and send environmental humidity information to the determination unit.

10. The biological information management system according to claim 1, further comprising:
a detection unit configured to detect a pulse rate and a heart sound of the subject.

11. The biological information management system according to claim 5, wherein in order to determine an abnormality in smell of the subject, the main body apparatus includes a smell generation unit configured to generate a smell for the subject.

12. The biological information management system according to claim 1, wherein the determination unit is configured to acquire external information from an information terminal via an information communication network.

13. The biological information management system according to claim 1, wherein the attachment unit includes an attachment sheet, the body temperature measurement unit and the body water measurement unit are disposed on the attachment sheet, and the attachment unit is configured to be attached to the underarm portion of the subject.

14. The biological information management system according to claim 1, wherein the determination unit is configured to output a determination result of whether or not there is a possibility that the subject develops the infection based on a comparison result obtained by comparing the body temperature of the subject with a determination value of the body temperature and a comparison result obtained by comparing the body water of the subject with a determination value of the body water.

15. A biological information management system comprising:
an attachment unit that includes a body temperature measurement unit configured to measure a body temperature of a subject and a body water measurement unit configured to measure body water of the subject, in which the body temperature measurement unit and the body water measurement unit are configured to be attached to the body of the subject;
a biological information transmission unit configured to transmit the body temperature of the subject measured by the body temperature measurement unit and the body water of the subject measured by the body water measurement unit to a determination unit;
the determination unit being configured to output a determination result of whether or not there is a possibility that the subject develops infection or heatstroke, based on the body temperature of the subject and the body water of the subject transmitted from the biological information transmission unit; and
a main body apparatus including the determination unit, wherein the biological information transmission unit is disposed in the attachment unit, the attachment unit has identification information for identifying the subject, and the identification information is sent to the determination unit of the main body apparatus by the biological information transmission unit.

16. A method for managing biological information comprising:
attaching a body temperature measurement unit and a body water measurement unit to an underarm portion of a subject;
measuring a body temperature of the subject on the underarm portion of the subject with a body temperature measurement unit;
measuring body water of the subject on the underarm portion of the subject with a body water measurement unit;
transmitting the body temperature measured by the body temperature measurement unit and the body water measured by the body water measurement unit to a determination unit from a biological information transmission unit; and
outputting a determination result of whether or not there is a possibility that the subject develops an infection or heatstroke, based on the body temperature of the subject and the body water of the subject transmitted from the biological information transmission unit.

17. The method according to claim 16, further comprising:
determining whether or not the subject develops dehydration in addition to the infection or the heatstroke.

18. The method according to claim 16, further comprising:
recording the body temperature of the subject and the body water of the subject with a recording unit, the recording unit including a primary information recording unit configured to record, as primary information obtained from the subject, the body temperature of the subject and the body water of the subject, a secondary information recording unit configured to record, as secondary information obtained from the subject, an abnormality in taste and smell of the subject, and presence or absence of cough and dyspnea, and a tertiary information recording unit configured to record, as tertiary information, an external environment of a local place where the subject is present; and
notifying the determination result in a case where the determination unit outputs the determination result indicating that there is a possibility that the subject develops the infection or the heatstroke.

19. The method according to claim 16, further comprising:
displaying the determination result in a case where the determination unit outputs the determination result indicating that there is a possibility that the subject develops the infection or the heatstroke.

20. The method according to claim 16, further comprising:
determining whether or not the subject has a fever and whether or not the body water of the subject decreases; and
determining whether or not there is an abnormality in taste/smell, whether or not there is cough/dyspnea, and whether or not there is numbness in arms or legs or muscle spasms.
